(19) United States
(12) Patent Application Publication
(10) Pub. No.: US 2026/0207892 A1
(43) Pub. Date: Jul. 23, 2026

(54) Urinary Catheter With Drainage Holes And Eyelets

(71) Applicant: Hollister Incorporated, Libertyville, IL (US)

(72) Inventors: Satwinder S. Panesar, Foxford (IE); David F. Pierce, Ballina (IE); David J. Farrell, Ballina (IE); Rory O’Brien, Galway (IE); Keith Dwyer, Ballina (IE)

(21) Appl. No.: 19/140,319

(22) PCT Filed: Dec. 1, 2023
(86) PCT No.: PCT/US2023/082053

§ 371 Date: Jun. 17, 2025

Related U.S. Application Data
(60) Provisional application No. 63/476,207, filed on Dec. 20, 2022.

(51) Int. Cl. A61M 25/00

(52) U.S. Cl. CPC A61M 25/007; A61M 2202/0014; A61M 2202/04; A61M 2210/1085; A61M 2210/1089

(57) ABSTRACT

Catheters that have at least one drainage hole (6, 7, 8, 12, 14, 16, 18, 20, 22, 24, 26, 28, 30, 32, 34, 36, 38, 40, 40, 42, 44, 46, 48, 54, 56, 58, 60, 62, 64, 66, 68, 69, 70, 72, 74, 76, 78, 80, 82, 84, 86, 88, 90, 92, 94, 96, 93, 95, 97) and at least one eyelet (E1, E2) on the proximal insertion end portion of the catheter.

### CROSS-REFERENCE TO RELATED APPLICATION

This application claims the benefit of U.S. Provisional Application Ser. No. 63/476,207, filed Dec. 20, 2022, the disclosure of which is hereby incorporated by reference in its entirety.

### FIELD OF THE DISCLOSURE

This disclosure relates generally to urinary catheters. More particularly, this disclosure relates to intermittent urinary catheters with at least one drainage hole in addition to at least one eyelet associated with the proximal insertion end portion of the catheter.

### BACKGROUND

Intermittent urinary catheters include a catheter shaft having a proximal insertion end portion that is inserted through the urethra and into the bladder. Once in the bladder, the urine enters the catheter through one or more openings. The urine enters into the opening(s) and drains through a drainage lumen of the catheter and out of a distal drainage opening in the distal end portion of the catheter shaft.

FIGS. 1 and 2 illustrate a prior art example of an intermittent urinary catheter. Catheter C includes an elongated catheter shaft S having a proximal insertion end portion P and a distal end portion D. Proximal insertion end portion P includes a terminal proximal end 100 that is suitable for insertion into the urethra. The proximal insertion end portion includes eyelets E1 and E2 for receiving urine there through and into an internal conduit or lumen of catheter shaft S. Distal end portion D may include a distal opening that is in fluid communication with a drainage member F, such as a funnel, for fluidly connecting catheter C to a collection container, such as a collection bag, or for directing urine to a waste receptacle, such as a toilet.

The catheter includes longitudinal axis A. Although the catheter is an elongated cylinder, there are a number of surfaces that can be defined in relation to the placement of the catheter eyelets. As shown in FIGS. 1 and 2, there is top surface 13, which is the surface of the first eyelet E1. There is also a second surface, or bottom surface 15, which is the surface of the second eyelet E2. There are two opposing surfaces, 17 and 19, respectively the left and right surfaces, that are on a perpendicular plane about the axis A from the top and bottom surfaces 13, 15 of the catheter.

The prior art catheter utilizes two eyelets for better draining during use. However, this draining may not be completely effective and may result in residue.

There remains a need for catheters with improved draining.

### SUMMARY

In a first aspect, an intermittent urinary catheter includes a catheter shaft having a proximal insertion end portion for advancement through a urethra into a bladder and a distal drainage end portion having a drainage opening. The catheter shaft has a drainage lumen in communication with the drainage opening. The intermittent urinary catheter includes at least one drainage hole and at least one eyelet located on the proximal insertion end portion. The at least one drainage hole has a diameter of 0.8 mm to about 1.56 mm and the at least one eyelet has a width from about 1.34 mm to about 2 mm and a length from about 1.5 mm to 5 mm.

In a second aspect, an intermittent urinary catheter includes a catheter shaft having a proximal insertion end portion for advancement through a urethra into a bladder and a distal drainage end portion having a drainage opening. The catheter shaft has a drainage lumen in communication with the drainage opening. The intermittent urinary catheter includes at least one drainage hole and at least one eyelet located on the proximal insertion end portion. The at least one drainage hole has a cross-sectional area from about 0.5 mm² to about 1.9 mm² and the at least one eyelet has a cross-sectional area from about 2 mm² to about 10 mm².

In another aspect, an intermittent urinary catheter includes a catheter shaft having a proximal insertion end portion for advancement through a urethra into a bladder and a distal drainage end portion having a drainage opening. The catheter shaft has a drainage lumen in communication with the drainage opening. The intermittent urinary catheter includes at least one drainage hole and at least one eyelet located on the proximal insertion end portion. A length or a width of the at least one eyelet is at least 2 times larger than a diameter of the at least one drainage hole.

In yet another aspect, an intermittent urinary catheter includes a catheter shaft having a proximal insertion end portion for advancement through a urethra into a bladder and a distal drainage end portion having a drainage opening. The catheter shaft has a drainage lumen in communication with the drainage opening. The intermittent urinary catheter includes at least one drainage hole and at least one eyelet located on the proximal insertion end portion. A cross-sectional area of the at least one eyelet is at least 4 times larger than a cross-sectional area of the at least one drainage hole.

In another aspect, an intermittent urinary catheter includes a catheter shaft having a proximal insertion end portion for advancement through a urethra into a bladder and a distal drainage end portion having a drainage opening. The catheter shaft has a drainage lumen in communication with the drainage opening. The intermittent urinary catheter includes at least one drainage hole and at least one eyelet located on the proximal insertion end portion. A flow rate of a liquid through the at least one eyelet plus at least one drainage hole is at least 1.1 times faster than a flow rate of a liquid through the at least one eyelet.

### BRIEF DESCRIPTION OF THE DRAWINGS

FIG. 1 is a perspective view of a prior art urinary catheter;

FIG. 2 is a side view of the proximal insertion end of the prior art urinary catheter of FIG. 1;

FIG. 3 is a side view of the proximal insertion end of a urinary catheter of an exemplary embodiment of the current disclosure;

FIG. 4 is a side view of the proximal insertion end of a urinary catheter of an exemplary embodiment of the current disclosure;

FIG. 5 is a side view of the proximal insertion end of a urinary catheter of an exemplary embodiment of the current disclosure;

FIG. 6 is a side view of the proximal insertion end of a urinary catheter of an exemplary embodiment of the current disclosure;

FIG. 7 is a side view of the proximal insertion end of a urinary catheter of an exemplary embodiment of the current disclosure;

FIG. 8 is a side view of the proximal insertion end of a urinary catheter of an exemplary embodiment of the current disclosure;

FIG. 9 is a side view of the proximal insertion end of a urinary catheter of an exemplary embodiment of the current disclosure;

FIG. 10 is a side view of the proximal insertion end of a urinary catheter of an exemplary embodiment of the current disclosure;

FIG. 11 is a side view of the proximal insertion end of a urinary catheter of an exemplary embodiment of the current disclosure;

FIG. 12 is a side view of the proximal insertion end of a urinary catheter of an exemplary embodiment of the current disclosure;

FIG. 13 is a side view of the proximal insertion end of a urinary catheter of an exemplary embodiment of the current disclosure;

FIG. 14 is a side view of the proximal insertion end of a urinary catheter of an exemplary embodiment of the current disclosure;

FIG. 15 is a side view of the proximal insertion end of a urinary catheter of an exemplary embodiment of the current disclosure;

FIG. 16 is a side view of the proximal insertion end of a urinary catheter of an exemplary embodiment of the current disclosure;

FIG. 17 is a side view of the proximal insertion end of a urinary catheter of an exemplary embodiment of the current disclosure;

FIG. 18 is a side view of the proximal insertion end of a urinary catheter of an exemplary embodiment of the current disclosure;

FIG. 20 is a side view of the proximal insertion end of a urinary catheter of an exemplary embodiment of the current disclosure;

FIG. 21 is a side view of the proximal insertion end of a urinary catheter of an exemplary embodiment of the current disclosure;

FIG. 22 is a side view of the proximal insertion end of a urinary catheter of an exemplary embodiment of the current disclosure;

FIG. 23 is a side view of the proximal insertion end of a urinary catheter of an exemplary embodiment of the current disclosure;

FIG. 24 is a side view of the proximal insertion end of a urinary catheter of an exemplary embodiment of the current disclosure;

FIG. 25 is a side view of the proximal insertion end of a urinary catheter of an exemplary embodiment of the current disclosure;

FIG. 26 is a side view of the proximal insertion end of a urinary catheter of an exemplary embodiment of the current disclosure; and

FIG. 27 is a side view of the proximal insertion end of a urinary catheter of an exemplary embodiment of the current disclosure.

### DETAILED DESCRIPTION OF THE EMBODIMENTS

The present disclosure is directed to intermittent urinary catheters having proximal insertion tips, wherein the urinary catheters include at least one eyelet and at least one drainage hole, which are positioned relatively close to the proximal terminal end of the catheter shaft.

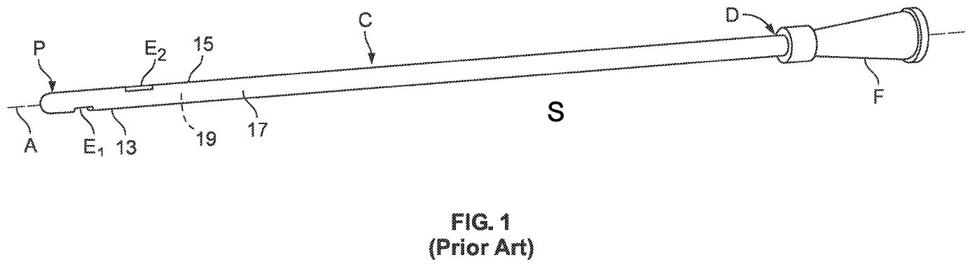

Turning now to FIGS. 3-27, a number of embodiments of urinary catheters of the present disclosure are shown. The figures include the proximal insertion end portion P of the catheters. Similar to FIG. 1, the urinary catheter may include a distal end portion (not shown). In the embodiments of the catheters disclosed herein, the distal end portion of the catheter shaft includes a drainage opening and, optionally, may include a drainage member (not shown) or be connected to a collection bag (not shown). Furthermore, the urinary catheter includes a drainage lumen that is in communication with the distal end drainage opening of the catheter shaft.

The proximal insertion end portion P includes a terminal proximal end 100 (labeled in FIG. 3). In the embodiments disclosed herein, the terminal proximal end 100 may be a closed end or an open end. When the terminal end 100 is an open end, the terminal end includes an opening in communication with drainage lumen.

The proximal insertion end portion P may also include at least one drainage hole and at least one eyelet. The drainage holes or holes may be positioned differently relative to the eyelet or eyelets in different embodiments of the current disclosure. In embodiments with a single eyelet, the drainage holes may be located proximately or distally from the eyelet relative to the terminal proximal end 100 of the catheter. In embodiments with two eyelets, as shown in FIGS. 3-27, the drainage hole or holes can be variously located. The drainage hole or holes may be located proximally from the eyelets, closer to the terminal proximal end 100 of the catheter. The drainage hole or holes may be located between the eyelets. The drainage holes may also be a combination of these locations, i.e., some of the holes proximal the most proximate eyelet, closer to the proximal end of the catheter and some of the holes between the two eyelets. The hole(s) may be located on the same surfaces as the eyelets or one at least one of the left and right surfaces 17 and 19 of the catheter.

The drainage hole(s) and eyelet(s) extend through the proximal insertion end portion P and in communication with the drainage lumen. The urinary catheter shaft includes a sidewall having an outer surface and an inner surface. The drainage hole(s) and eyelet(s) extend through the sidewall from the outer surface to the inner surface. The outer surface may have a hydrophilic coating disposed thereon.

The proximal most drainage hole or eyelet may be located between about 2 mm and about 50 mm, or preferably between about 5 mm and about 40 mm from the proximal terminal end 100 of the catheter.

The drainage holes and eyelets of the embodiments disclosed herein may be formed in any suitable manner, including but not limited to, mechanical punching, thermal forming, ultrasonic cutting, laser cutting, etc. The drainage holes and eyelets in the embodiments disclosed herein are arranged to maximize the drainage ability and efficiency.

In the illustrated embodiments (except for the slit embodiments), each of the drainage holes is generally the same size and shape. For instance, the drainage holes have a generally round shape. However, in other embodiments, the drainage holes may have other shapes, such as oval, oblong, polygonal or irregular. In alternative embodiments, the drainage holes may have various sizes and shapes relative to one another.

In the illustrated embodiment, the eyelets have a generally oval shape. However, in other embodiments, the eyelets may have other shapes, such as oval, circular, or polygonal. The eyelets may have any of the sizes described above. In alternative embodiments, the eyelets may have various sizes and shapes relative to one another.

The drainage holes and eyelets both function to drain fluid from the surface of the catheter to the inner lumen of the catheter. The eyelets are notably larger in size when compared to the drainage holes. The eyelets and drainage holes disclosed herein each include an opening or passageway that extends through and is bounded by the catheter wall. The cross-sectional area of the opening of the eyelet taken at any location along the catheter wall defining the opening is at least 2 mm². The cross-sectional area of eyelet may be from 2 mm² to 10 mm². The cross-sectional area of the opening of the drainage holes taken at any location along the catheter wall defining the opening is greater than 0.45 mm² and at most 1.9 mm². In one alternative, the cross-sectional area of the drainage hole is at least 0.5 mm² and at most is 1.9 mm². In one alternative, a single drainage hole has a diameter of at least 0.8 mm and at most 1.56 mm. In another alternative, a single drainage hole may have a square shape with where in sides have a length from about 0.89 mm to about 1.38 mm. In another alternative, a single drainage hole may have a rectangular shape with a width from about 0.04 mm to about 0.8 mm and length from about 1.1 mm to 4 mm and the cross-sectional area of the opening is at least 0.5 mm² and at most is 1.9mm². The ratio of the cross-sectional area of an eyelet's opening to the cross-sectional area of a single drainage hole's opening is from 20:1 and can be 1.1:1. Alternatively, or in addition to, the relative sizes, the flow rate of fluid through a drainage hole is about 0.95 to 1/20 the flow rate of fluid through the eyelet. Optionally, the smallest cross-sectional length (e.g., diameter) of the drainage holes taken from one point D of the catheter sidewall defining the hole to a direct opposite point D′ on the catheter sidewall can range from about 0.8 mm to about 1.6 mm, and/or the largest cross-sectional length of the eyelets taken from one point D″ of the catheter sidewall defining the eyelet to a direct opposite point D″′ on the catheter sidewall can range from 1.5 mm to 5 mm. An eyelet may have a width from about 1.34 mm to about 2.0 mm and a length from about 1.5 mm to about 5 mm. In comparison, a length or a width of an eyelet is at least 2 times the diameter of a drainage hole, but can be 4 times larger or even 6 times larger than the diameter of a drainage hole. Additionally, the cross-sectional area of an eyelet is at least 4 times larger than the cross-sectional area of a drainage hole, but can be 5 times larger or even 6 times larger than the cross-sectional area of a drainage hole.

Additional drainage holes will improve flow rate through lumen of the catheter. The flow rate of a liquid through the at least one eyelet plus one drainage hole is at least 1.1 times faster than the rate of a liquid through the at least one eyelet. With the presence of multi drainage holes the flow rate through lumen is at least 1.2 times faster than the rate of a liquid through the at least 2 drainage hole.

A number of different eyelet(s) and drainage hole(s) arrangements are disclosed in FIGS. 3-27. These are non-limiting and other arrangements are within the scope of this disclosure.

FIG. 3 shows a possible embodiment with a single drainage hole 6 arranged proximally from the first eyelet E1, toward the terminal proximal end of the catheter. Hole 6 is arranged on an opposite surface of the catheter from the eyelet E1 and on the same surface as the eyelet E2 determined from the longitudinal axis A of the catheter. In the illustrated embodiment, eyelet E1 is in the top surface 13 of the catheter. Hole 6 and E2 are arranged on the bottom surface 15 of the catheter as determined from the longitudinal axis A of the catheter.

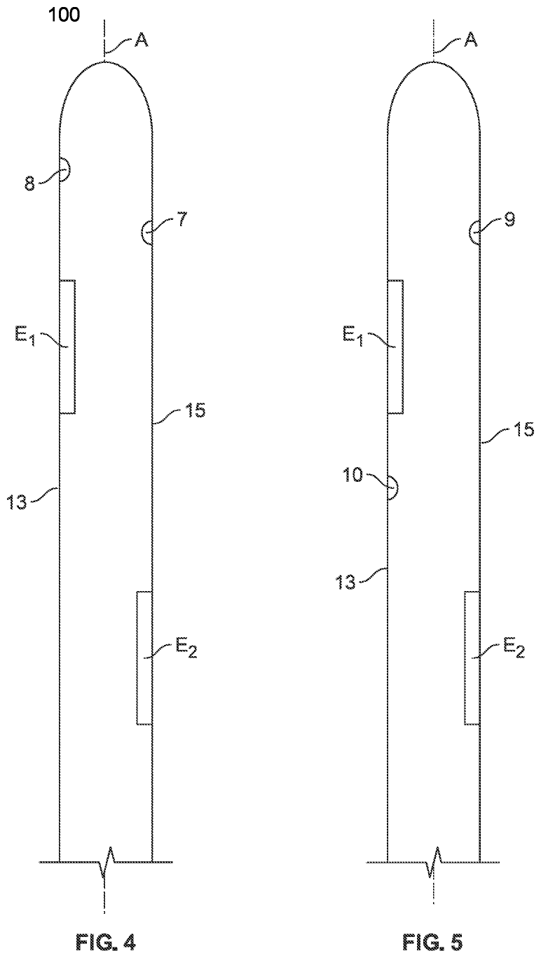

FIG. 4 includes two drainage holes 7, 8, which are located proximal of the first eyelet E1 toward the proximal end of the catheter. The first eyelet E1 being the proximal most eyelet or the eyelet closest to the terminal end 100. The two drainage holes are located on opposite longitudinal surfaces of the catheter, with one drainage hole 8 on the same surface as the first eyelet E1, or the top surface 13, and the other drainage hole 7 on the same surface as the eyelet E2, or the bottom surface 15. Although shown in FIG. 4 as drainage hole 8 on the same surface as eyelet E1, the drainage holes 7 and 8 may be arranged opposite so that hole 7 is more proximal and located toward the proximal end of the catheter.

FIG. 5 includes two drainage holes, 9, 10 which are located in two different locations, one proximal the first eyelet E1, and one between the first and second eyelets E1 and E2. Drainage hole 9 may be on surface 15, the same surface as eyelet E2 and drainage hole 10 may be on the same longitudinal surface as the first eyelet E1. Alternatively, drainage hole 9 may be on the same longitudinal surface as E1 and drainage hole 10 may be on the opposite surface, the same surface as eyelet E2.

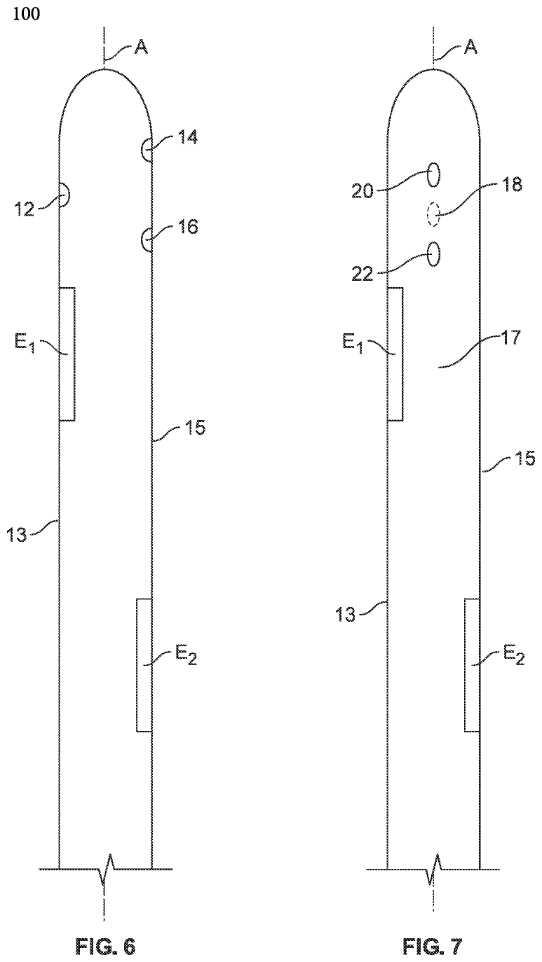

FIG. 6 includes three drainage holes 12, 14, and 16. All three drainage holes are located proximal of the first eyelet E1, which is the proximal most eyelet. In other words, all the drainage holes are located between eyelet E1 and the terminal proximal end 100. In the Figure they are arranged so that a single hole 12 is on the same top surface 13 as the first eyelet E1 and that hole is located vertically between two additional holes 14, 16 which are on the same surface 15 as second eyelet E2. Alternately, holes 14, 16 may be on the same surface 13 as first eyelet E1 and single hole 12 may be on the same surface 15 as second eyelet E2.

FIG. 7 includes three drainage holes 18, 20, 22. Drainage holes 20 and 22 are above and below drainage hole 18 and on opposite surfaces from drainage hole 18. As shown in the embodiment the top and bottom holes may be on the left surface 17 of the catheter tube and the middle hole 18 may be on the opposite, or right surface of the catheter. It is also possible for the two holes 20, 22 to be on the right surface 19 and the middle hole 18 to be on the left surface 17 of the catheter tube.

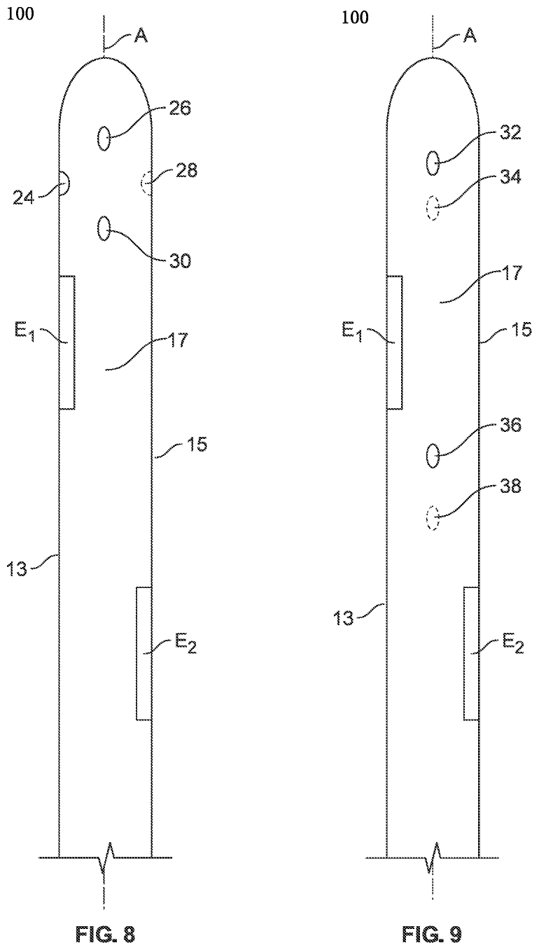

FIG. 8 includes four drainage holes 24, 26, 28 and 30 which are located proximal of the first eyelet E1, which is the proximal most eyelet. The drainage holes are located between E1 and the terminal proximal end 100. As shown in the FIG. 8, hole 24 may be on the same surface 13 of the catheter as E1 and another hole 28 may be on the same surface of the catheter as the second eyelet E2. These holes may be between longitudinally spaced from the other two holes, 26 and 30. The other two holes 26, 30 are shown on the left side 17 of the catheter. The two holes 26 and 30 may also be both arranged on the right side of the catheter. Although 26 and 30 are shown on the same surface, they may be on opposing surfaces. Additionally, holes 26 and 30 may be on one of the same surfaces as either eyelet E1, E2 and the additional holes 24 and 28 may be the left and right side surfaces of the catheter.

FIG. 9 includes four drainage holes 32, 34, 36, 38. Two drainage holes 32, 34 may be proximal to the first eyelet E1, closer to the terminal proximal end 100 of the catheter. Two holes 36, 38 may be between the first and second eyelet E1 and E2. The holes 32, 34 are on the opposing left side surface 17 and right-side surface, different from the surfaces in which first or second eyelet E1 or E2 are located. Hole 32 is shown more proximal or closer to the terminal proximal end 100 of the catheter, but their positions relative to each other along the length of the catheter may be reversed. Drainage holes 36, 38 are on the opposing left and right-side surfaces, different from the surfaces in which the first or second eyelet E1 or E2 are located. Hole 36 is shown more proximal or closer to the terminal proximal end 100 of the catheter, but their longitudinal positions along the catheter may be reversed.

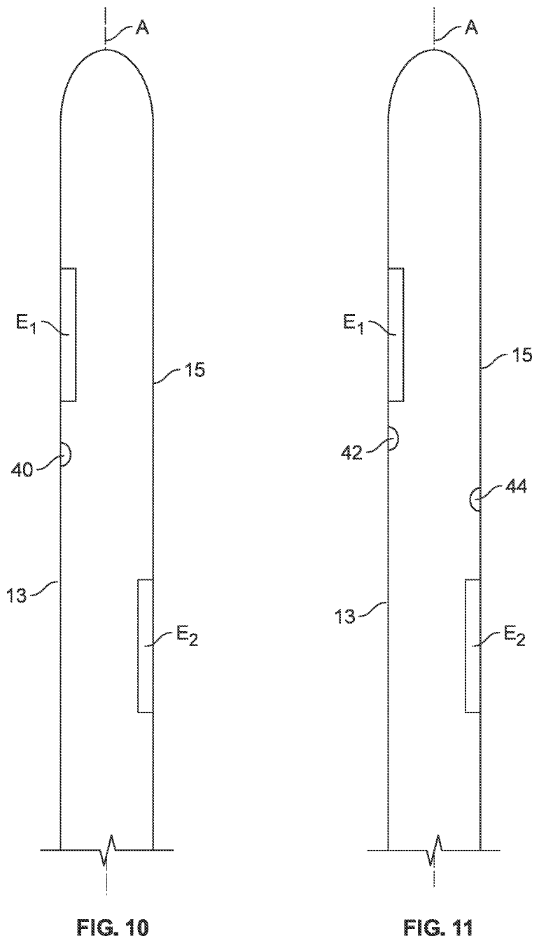

FIG. 10 shows a catheter that includes one drainage hole 40 placed between eyelets E1 and E2. As shown, the hole is closer to eyelet E1 and on the same surface as E1, but the hole may be closer to E2 and on the same surface as E2. Alternatively, hole 40 may be on one of the left or right surfaces of the catheter.

FIG. 11 shows a catheter that includes two drainage holes 42, 44. Both holes are placed between eyelets E1 and E2. As shown in the figure, hole 42 may be on the same surface as E1 and hole 44 may be on the same surface as E2, therefore on opposing surfaces of the catheter. Alternatively, the holes 42, 44 can be on alternate opposing left and right surfaces. Hole 42 is shown as distal to and closer to E1 and hole 44 as proximal and closer to E2. However, this may be alternately reversed so that hole 42 is closer to E2 and hole 44 is closer to E1.

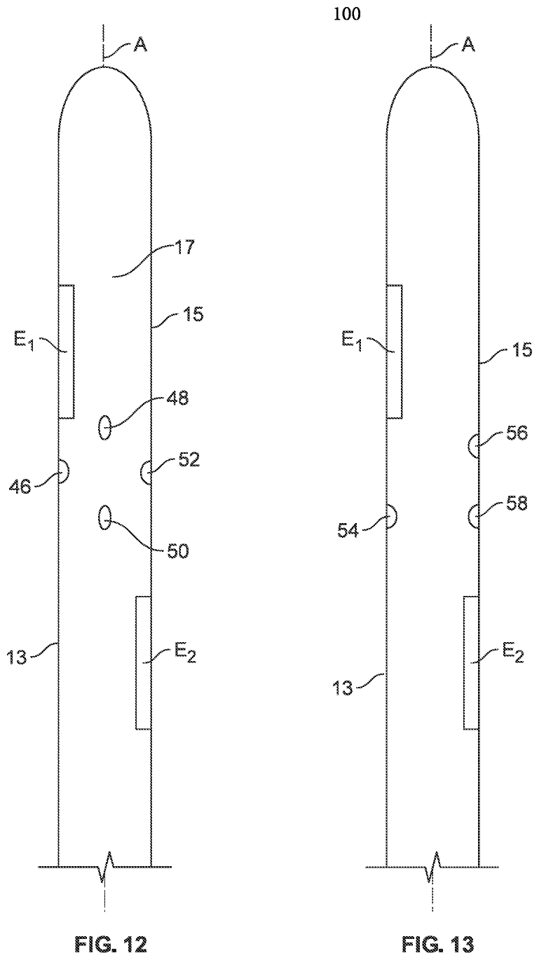

FIG. 12 shows a catheter that includes four drainage holes 46, 48, 50, 52, which may be in a clover pattern. The four drainage holes are located between eyelets E1 and E2. Holes 46 and 52 are located at the same longitudinal position on the catheter, and between holes 48 and 50. Holes 46 and 52 are one of the surfaces of the eyelets E1 and E2, respectively. Holes 48 and 50 are located on the left surface 17 of the catheter, but may alternately be on the right surface of the catheter. Alternatively, although 48 and 50 are shown on the same surface, they may be on opposing surfaces.

FIG. 13 includes three drainage holes 54, 56, and 58. All three holes are placed between eyelets E1 and E2. Holes 54 and 58 are shown at the same approximate height and on opposing surfaces 13, 15, one on each of the surfaces of eyelets E1 and E2. Hole 56 is shown in a slightly more proximal location from 54 and 58, closer to E1 and the terminal proximal end 100 of the catheter. Hole 56 is shown as being on the same surface as hole 58, the surface of eyelet E2. However, hole 56 may be placed on the same surface as hole 54 and eyelet E1. Additionally, hole 56 may be on a different surface, such as the left or right surface 17, 19 of the catheter.

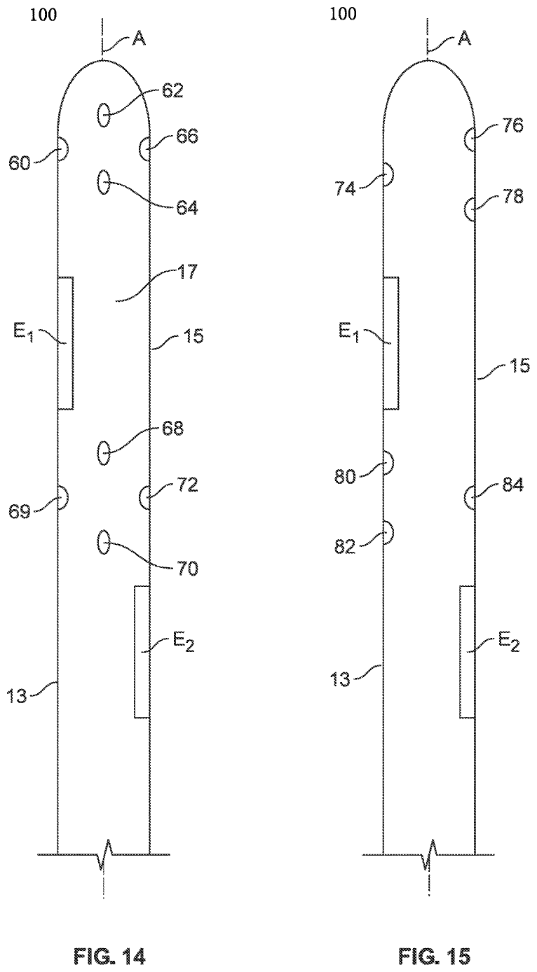

FIG. 14 includes eight drainage holes in two sets of four, 60, 62, 64, and 66 and 68, 69, 70 and 72. Each set may be in a clover pattern. Holes 60, 62, 64, and 66 are higher or more proximal to the terminal proximal end 100, compared to eyelet E1. Holes 68, 69, 70 and 72 are placed at a longitudinal position along the catheter between eyelets E1 and E2. As shown in the FIG. 14, holes 60 and 66 are on opposite surfaces, each one on a surface of eyelet E1 or eyelet E2. Holes 69 and 72 are also on opposite surfaces, each one on a surface of eyelet E1 or eyelet E2. The holes 60 and 66 and 69 and 72 are in parallel positions on opposite surfaces. These holes are between the pairs of holes 62, 64 and 68, 70. As shown in FIG. 14, the holes may be on the same surface, the left surface 17 of the catheter. Although the pair of holes 62, 64 and 68, 70 are shown on the same surface, it is alternately possible that each hole is on an opposing surface or that all of the holes are on the right surface of the catheter.

FIG. 15 includes six drainage holes 74, 76, 78, 80, 82 and 84. The holes are divided into two groups of three holes. The first group of holes 74, 76, and 78 is positioned proximal of the first eyelet E1, more proximate to the terminal proximal end 100 of the catheter. The second group of holes are positioned between the first and second eyelets E1 and E2. The first group of holes 74, 76 and 78 include a first hole positioned on the same surface 13 of the catheter as eyelet E1 and two holes 76 and 78 positioned on the opposite surface 15, the same catheter surface as eyelet E2. Hole 74 is positioned between the holes 76 and 78. Alternatively the holes may be on opposite surfaces as shown and may also be on different opposite surfaces, such as the left and right surfaces of the catheter. The second group of holes 80, 82 and 84 include a first hole 84 positioned on the same surface of the catheter as eyelet E2 and two holes 80 and 82 positioned on the opposite surface, the same catheter surface as eyelet E1. Hole 84 is positioned between the holes 80 and 82. Alternatively the holes may be on opposite surfaces as shown and may also be on different opposite surfaces, such as the left and right surfaces of the catheter.

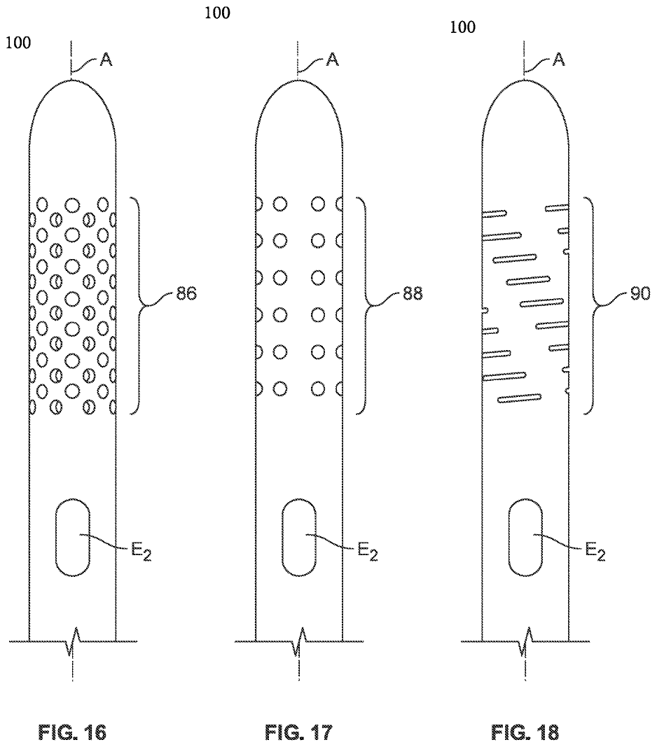

FIG. 16 includes a group of drainage holes 86 between eyelet E2 and the terminal proximal end 100 of the catheter. In the illustrated embodiment, the drainage holes 86 are arranged in a plurality of columns. Each column has an axis parallel to the longitudinal axis A of the catheter shaft. In the illustrated embodiment, the columns C are offset or in a variable pattern. The drainage holes pattern is present around the entire catheter.

FIG. 17 includes a group of drainage holes 88 between eyelet E2 and the terminal proximal end 100 of the catheter. In the illustrated embodiment, the drainage holes are arranged in a plurality of identical, equally spaced columns. The drainage holes pattern is present on the entire circumference of the catheter.

FIG. 18 includes a group of drainage holes 90 between eyelet E2 and the terminal proximal end 100 of the catheter. In the illustrated embodiment, the drainage holes 90 are slots. In this embodiment, the slots are patterned in a helical configuration about axis A of the catheter shaft. In the illustrated embodiment, the drainage holes 90 are elongated slits having an elongated arc or axis that extends partially along the outer circumference of the catheter shaft. The drainage holes 90 may have a length of 1.5 mm to 3 mm, preferably 2 mm to 2.6 mm preferably 2.3 mm and a width of 0.1 to 0.6 mm and preferably 0.15 to 0.3 mm and preferably 0.2 mm. Additionally, the slits may be aligned in offset columns. The columns have an axis that is parallel with the axis A of the catheter shaft. The group of drainage holes 90 replace eyelet E1 of the prior art catheter.

FIG. 19 includes a group of drainage holes 92. In the illustrated embodiment, the drainage holes 92 are arranged in a plurality of columns. Each column has an axis parallel to the longitudinal axis A of the catheter shaft. In the illustrated embodiment, the columns are offset or in a variable pattern. Eyelets E1 and E2 are still included on the catheter, but below the group of drainage holes 92, drainage holes 92 are more proximate to the terminal proximal end 100 of the catheter. In other words, the drainage holes are between the most proximal eyelet E1 and the terminal proximal end 100 of the catheter. The drainage holes pattern is present along the entire circumference of the catheter. Eyelets E1 and E2 may be closer together than in previous embodiments (8-9 mm) or may be spaced similarly.

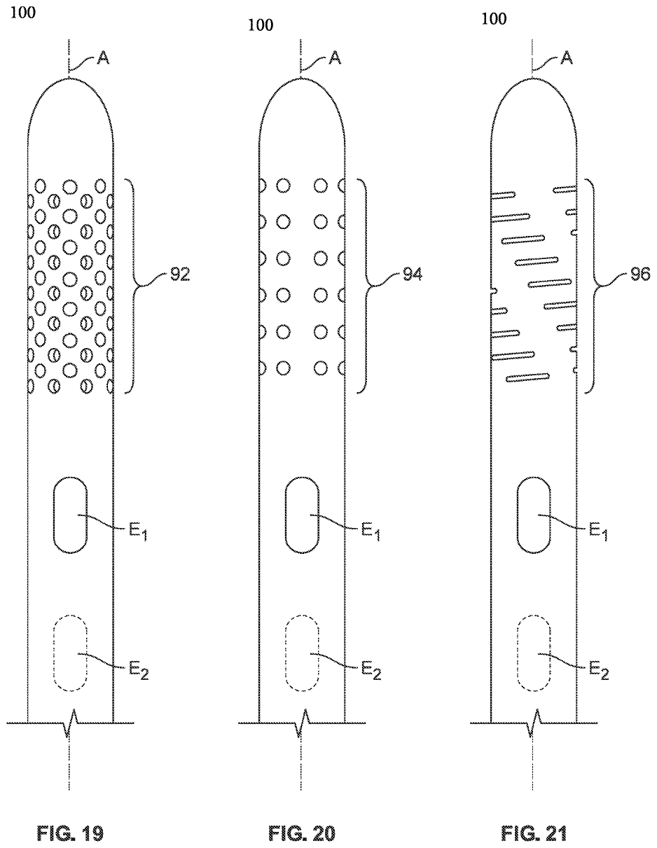

FIG. 20 includes a group of drainage holes 94. In the illustrated embodiment, the drainage holes are arranged in a plurality of identical, equally spaced columns. Eyelets E1 and E2 are still included on the catheter, but below the group of drainage holes 94, drainage holes 94 are more proximate to the proximal end of the catheter 100. In other words, the drainage holes are between the most proximal eyelet E1 and the terminal proximal end 100 of the catheter. The drainage holes pattern is present along the entire circumference of the catheter. Eyelets E1 and E2 may be closer to together than in previous embodiments (8-9 mm) or similarly spaced.

FIG. 21 includes a group of drainage holes 96. In the illustrated embodiment, the drainage holes 96 are slots. In this embodiment, the slots are patterned in a helical configuration about axis A of the catheter shaft. In the illustrated embodiment, the drainage holes 96 are elongated slits having an elongated arc or axis that extends partially along the outer circumference of the catheter shaft. The drainage holes 96 may have a length of 1.5 mm to 3 mm, preferably 2 mm to 2.6 mm preferably 2.3 mm and a width of 0.1 to 0.6 mm, preferably 0.15 mm to 0.3 mm and preferably 0.2 mm. Eyelets E1 and E2 are still included on the catheter, but below the group of drainage holes 96, drainage holes 96 are more proximate to the terminal proximal end of the catheter 100. In other words, the drainage holes are between the most proximal eyelet E1 and the terminal proximal end 100 of the catheter. The drainage holes pattern is present along the entire circumference of the catheter. Eyelets E1 and E2 may be closer to together than in previous embodiments (8-9 mm) or similarly spaced.

FIG. 22 includes a group of drainage holes 93. In the illustrated embodiment, the drainage holes 93 are arranged in a plurality of columns. Each column has an axis parallel to the longitudinal axis A of the catheter shaft. In the illustrated embodiment, the columns are offset or in a variable pattern. The drainage holes pattern does not extend around the entire circumference of the catheter. The center column is on the opposite surface 15 of E1 and the offset columns extend radially from this center column. The drainage holes extend from the left surface of the catheter to the right surface of the catheter, across the bottom surface 15 of the catheter. E2 is still located on the bottom surface 15 of the catheter, the same surface as the drainage holes 93.

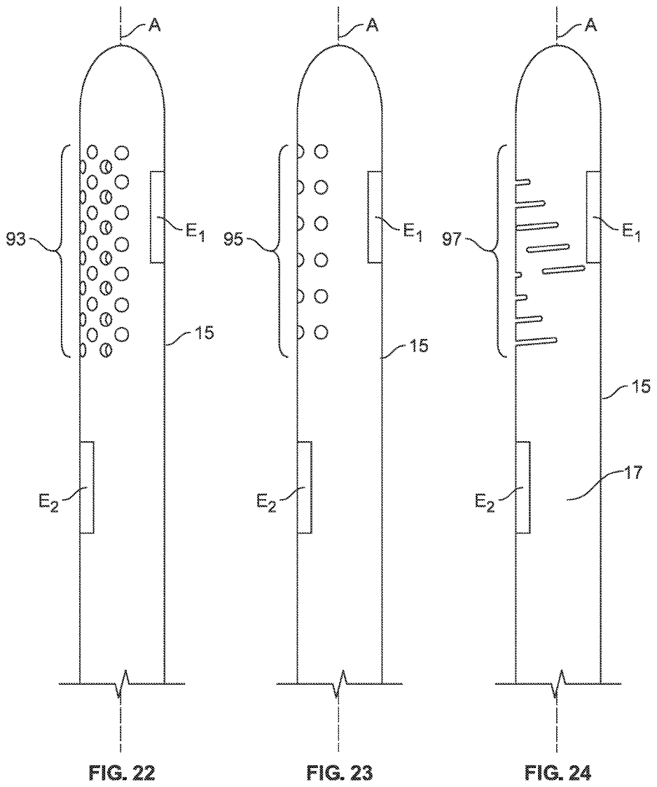

FIG. 23 includes a group of drainage holes 95. In the illustrated embodiment, the drainage holes are arranged in a plurality of identical, equally spaced columns. The drainage holes pattern does not extend around the entire circumference of the catheter. The center column is on the opposite surface of E1, and the columns extend radially from this center column. E2 is still located on the bottom surface 15 of the catheter, the same surface as the drainage holes 95.

FIG. 24 includes a group of drainage holes 97. In the illustrated embodiment, the drainage holes 97 are slots. In this embodiment, the slots are patterned in a helical configuration about axis A of the catheter shaft. In the illustrated embodiment, the drainage holes 97 are elongated slits having an elongated arc or axis that extends partially along the outer circumference of the catheter shaft. The drainage holes 97 may have a length of 1.5 mm to 3 mm, 2 mm to 2.6 mm, preferably 2.3 mm and a width of 0.1 to 0.6 mm, preferably 0.15 mm to 0.3 mm and preferably 0.2 mm. Additionally, the slits may be aligned in offset columns. The drainage holes 97 do not extend around the entire circumference of the catheter. The drainage holes extend from the left surface 17 of the catheter to the right surface 19 of the catheter, across the bottom surface 15 of the catheter, opposite eyelet E1. E2 is still located on the bottom surface 15 of the catheter, the same surface as the drainage holes 97.

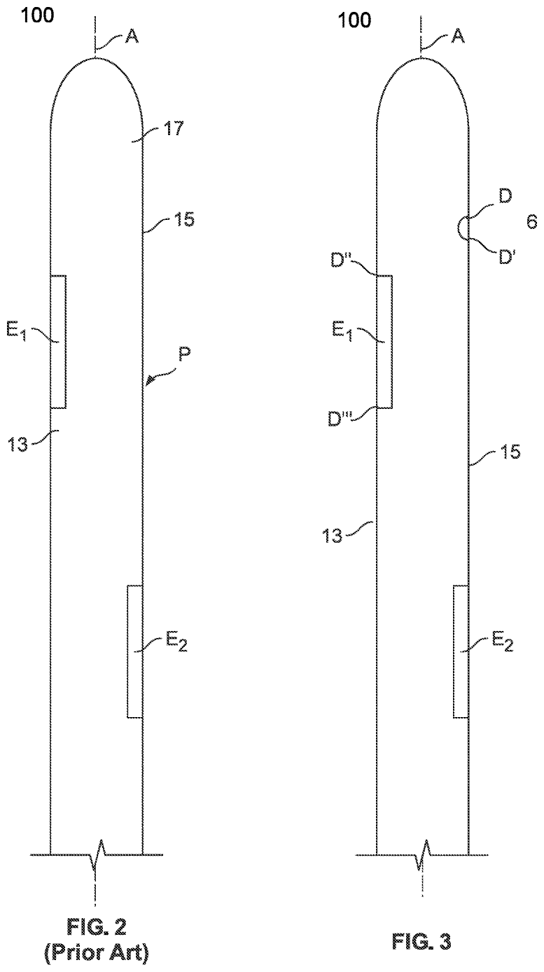

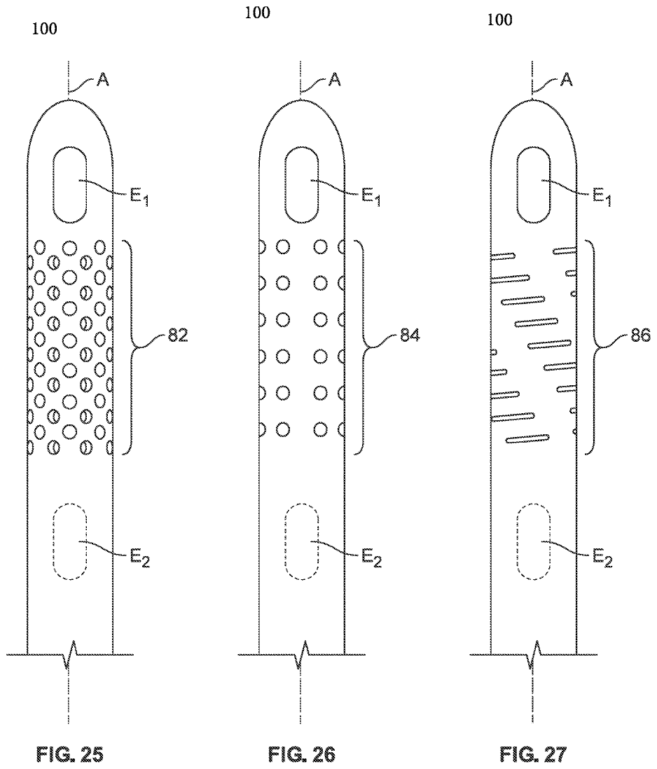

FIG. 25 includes a group of drainage holes 82. In the illustrated embodiment, the drainage holes 82 are arranged in a plurality of columns. Each column has an axis parallel to the longitudinal axis A of the catheter shaft. In the illustrated embodiment, the columns are offset or in a variable pattern. The drainage hole pattern extends around the entire circumference of the catheter. Eyelets E1 and E2 are still included on the catheter, the group of drainage holes 82 are between the eyelets E1 and E2. Eyelets E1 and E2 may be in the same relative location as FIG. 2 and Eyelet E1 is 10 mm from the terminal proximal end 100, or Eyelet E1 may be vertically higher or closer to the terminal proximal end 100, less than 10 mm the terminal proximal end 100 Also the distance between Eyelets E1 and E2 is 8 mm to 9 mm, or greater than 8 mm to 9 mm.

FIG. 26 includes a group of drainage holes 84. In the illustrated embodiment, the drainage holes are arranged in a plurality of identical, equally spaced columns. The drainage hole pattern extends around the entire circumference of the catheter. Eyelets E1 and E2 are still included on the catheter, the group of drainage holes 84 are between the eyelets E1 and E2. Eyelets E1 and E2 may be in the same relative location as FIG. 2, or Eyelet E1 may be vertically higher or closer to the terminal proximal end 100.

FIG. 27 includes a group of drainage holes 86. In the illustrated embodiment, the drainage holes 86 are slots. In this embodiment, the slots are patterned in a helical configuration about axis A of the catheter shaft. In the illustrated embodiment, the drainage holes 86 are elongated slits having an elongated arc or axis that extends partially along the outer circumference of the catheter shaft. The drainage holes 86 may have a length of 2 mm to 3 mm, preferably 2.6 mm and a width of 0.1 to 0.3 mm and preferably 0.2 mm. Additionally, the slits may be aligned in offset columns. The drainage hole pattern extends around the entire circumference of the catheter. Eyelets E1 and E2 are still included on the catheter, the group of drainage holes 86 are between the eyelets E1 and E2. Eyelets E1 and E2 may be in the same relative location as FIG. 2, or Eyelet E1 may be vertically higher or closer to the terminal proximal end 100.

It should be understood that various changes and modifications to the presently preferred embodiments described herein will be apparent to those skilled in the art. Such changes and modifications can be made without departing from the spirit and scope of the invention disclosed herein.

What is claimed is:

1. An intermittent urinary catheter, comprising:
a catheter shaft having a proximal insertion end portion for advancement through a urethra into a bladder and a distal drainage end portion having a drainage opening, the catheter shaft having a drainage lumen in communication with the drainage opening; and
at least one drainage hole and at least one eyelet located on the proximal insertion end portion;
wherein the at least one drainage hole has a diameter of 0.8 mm to about 1.56 mm and the at least one eyelet has a width from about 1.34 mm to about 2 mm and a length from about 1.5 mm to 5 mm.

2. An intermittent urinary catheter, comprising:
a catheter shaft having a proximal insertion end portion for advancement through a urethra into a bladder and a distal drainage end portion having a drainage opening, the catheter shaft having a drainage lumen in communication with the drainage opening; and
at least one drainage hole and at least one eyelet located on the proximal insertion end portion;
wherein the at least one drainage hole has a cross-sectional area from about 0.5 mm² to about 1.9 mm² and the at least one eyelet has a cross-sectional area from about 2 mm² to about 10 mm².

3. An intermittent urinary catheter, comprising:
a catheter shaft having a proximal insertion end portion for advancement through a urethra into a bladder and a distal drainage end portion having a drainage opening, the catheter shaft having a drainage lumen in communication with the drainage opening; and
at least one drainage hole and at least one eyelet located on the proximal insertion end portion;
wherein a length or a width of the at least one eyelet is at least 2 times larger than a diameter of the at least one drainage hole.

4. (canceled)

5. (canceled)

6. The intermittent urinary catheter of claim 1, wherein the intermittent urinary catheter includes at least two eyelets.

7. The intermittent urinary catheter of claim 6, wherein the at least one drainage hole is located between the two eyelets.

8. The intermittent urinary catheter of claim 6, wherein the at least one drainage hole is located proximal of the two eyelets.

9. The intermittent urinary catheter of claim 1, wherein the intermittent urinary catheter includes at least two drainage holes.

10. The intermittent urinary catheter of claim 1, wherein the intermittent urinary catheter includes at least four drainage holes.

11. The intermittent urinary catheter of claim 1, wherein the at least one drainage hole is at least two holes and the at least one eyelet is two eyelets including a first eyelet and a second eyelet, and the holes are divided into groups of at least one hole, a first group proximal of the first eyelet and a second group located between the first and the second eyelet.

12. The intermittent urinary catheter of claim 1, wherein the at least one drainage hole or the at least one eyelet is located between 2 mm and 30 mm, or preferably between 5 mm and 15 mm from a terminal proximal end.

13. The intermittent urinary catheter of claim 1, wherein the at least one drainage hole comprises a plurality of drainage holes arranged in a pattern.

14. The intermittent urinary catheter of claim 13, wherein the pattern of the plurality of drainage holes comprises the drainage holes being arranged in a plurality of columns, each column having an axis parallel to a longitudinal axis of the catheter shaft.

15. The intermittent urinary catheter of claim 14, wherein each column of drainage holes is a same distance from adjacent columns.

16. The intermittent urinary catheter of claim 13, wherein the drainage holes comprise elongated slots.

17. The intermittent urinary catheter of claim 16, the pattern of the plurality of drainage holes comprises elongated slots extending in a helical pattern about the catheter shaft.

18. The intermittent urinary catheter of claim 13, wherein the pattern of the plurality of drainage holes extends around an entire circumference of the intermittent urinary catheter.

19. The intermittent urinary catheter of claim 13, wherein the pattern of the plurality of drainage holes extends around a portion of a circumference of the intermittent urinary catheter.

20. The intermittent urinary catheter of claim 13, wherein the at least one eyelet includes a first and second eyelet.

21. The intermittent urinary catheter of claim 20, wherein the drainage holes are located proximally of both eyelets.

22. The intermittent urinary catheter of claim 20, wherein the drainage holes are located between the first and second eyelet.

23. (canceled)

24. (canceled)
